(19) United States
(12) Patent Application Publication
(10) Pub. No.: US 2021/0252236 A1
(43) Pub. Date: Aug. 19, 2021

(54) RESPIRATORY THERAPY DEVICES AND ASSEMBLIES

(71) Applicant: SMITHS MEDICAL INTERNATIONAL LIMITED, Ashford (GB)

(72) Inventors: Mohammad Qassim Mohammad Khasawneh, Canterbury (GB); Steven Mark Tupper, Hythe (GB); Usamah Hanif, Ashford (GB)

(73) Assignee: SMITHS MEDICAL INTERNATIONAL LIMITED, Ashford (GB)

(21) Appl. No.: 17/251,323

(22) PCT Filed: Jun. 13, 2019
(86) PCT No.: PCT/GB2019/000083

§ 371 Date: Dec. 11, 2020

(30) Foreign Application Priority Data
Jun. 21, 2018 (GB) 1810197.2

(51) Int. Cl. A61M 11/06; A61M 16/20; A61M 16/08; A61M 16/00

(52) U.S. Cl. CPC A61M 11/06; A61M 16/0006; A61M 16/0816; A61M 16/209

(57) ABSTRACT

An oscillatory therapy device has a nebulizer port (21) close to a patient inlet (12) and a first one way valve (31) between the nebulizer port and the oscillatory mechanism (20) arranged to allow expiratory flow from the patient inlet to the mechanism but to prevent flow in the opposite direction. The nebulizer port (21) includes a second one-way valve (32) arranged to open during inhalation but to close during exhalation so that nebulizer medication is not expelled during exhalation.

This invention relates to oscillatory respiratory therapy devices of the kind with a patient inlet and a mechanism arranged to produce an oscillating resistance to breathing through the inlet.

Positive expiratory pressure (PEP) devices, that is, devices that present a resistance to expiration through the device, are now widely used to help treat patients suffering from a range of respiratory impairments, such as chronic obstructive pulmonary disease, bronchitis, cystic fibrosis and atelectasis. More recently, such devices that provide an alternating resistance to flow have been found to be particularly effective. One example of such a device is sold under the trade mark Acapella (a registered trade mark of Smiths Medical) by Smiths Medical and is described in U.S. Pat. Nos. 6,581,598, 6,776,159, 7,059,324 and 7,699,054. Other vibratory respiratory therapy devices are available, such as “Quake” manufactured by Thayer, “AeroPEP” manufactured by Monaghan, and “IPV Percussionator” manufactured by Percussionaire Corp. Alternative devices such as “CoughAssist” manufactured by Philips are also available.

Some patients having vibratory PEP therapy benefit from receiving nebulizer treatment such as with a bronchodilator medicine such as salbutamol. This nebulizer treatment can conveniently be given using the respiratory therapy by means of a T-piece adaptor fitted between the patient inlet and the mouthpiece of the therapy device. Alternatively, the therapy device could have a nebulizer coupling incorporated into the housing of the device as described in U.S. Pat. No. 8,225,785. In this way, when the patient exhales through the device he benefits from the vibratory resistance to exhalation and, when he inhales he receives a mixture of air and the nebulization vapour. One problem with prior arrangements is that the nebulization vapour is administered continuously both when the user inhales and when he exhales. This results in much of the nebulization medicine being wasted since some condenses on the inside surfaces of the therapy device and some is discharged to atmosphere during exhalation without having been inhaled into the user's respiratory passages.

It is an object of the present invention to provide an alternative oscillatory respiratory therapy device.

According to one aspect of the present invention there is provided an oscillatory respiratory therapy device of the above-specified kind, characterised in that the device includes a first one-way valve located between the patient inlet and the mechanism and arranged to allow expiratory flow from the patient inlet to the mechanism but to restrict flow through the valve in the opposite direction, that the device also includes a nebulizer port opening into the device between the patent inlet and the first valve, that the nebulizer port is adapted for connection with the outlet of a nebulizer, and that the device includes a second one-way valve arranged to allow flow from the port to the patient inlet during inhalation but to restrict flow from the port during exhalation.

The mechanism may include a displaceable member in the form of a pivoted rocker arm supporting a valve member that closes and opens an opening through which expiratory air flows. The first and second valves may be duck-bill valves. The second valve may be provided on a coupling separate from and removably connected to the therapy device, and the coupling may provide a connection for the outlet of a nebulizer. The coupling may also provide a connection for a patient interface.

According to another aspect of the present invention there is provided an assembly of a respiratory therapy device according to the above one aspect of the present invention and a nebulizer connected with the nebulizer port.

According to a further aspect of the present invention there is provided an assembly of a nebulizer and an oscillatory respiratory therapy device, the therapy device having a patient inlet and a mechanism arranged to produce an oscillating resistance to breathing through the inlet, characterised in that the assembly includes a first one-way valve located between the patient inlet and the mechanism and arranged to allow expiratory flow from the patient inlet to the mechanism but to restrict flow through the valve in the opposite direction, that the assembly also includes a nebulizer port opening into the device between the patent inlet and the first valve, that the nebulizer port is connected with the outlet of a nebulizer, and that the assembly includes a second one-way valve arranged to allow flow from the nebulizer to the patient inlet during inhalation but to restrict flow from the nebulizer during exhalation.

According to a fourth aspect of the present invention there is provided a nebulizer coupling for fitting to the patient inlet of an oscillatory respiratory therapy device, characterised in that the coupling includes a one-way valve arranged to allow flow from the coupling to the patient inlet during inhalation but to restrict flow from the coupling during exhalation.

The coupling may also provide a connection for a patient interface.

An oscillatory PEP therapy device and a nebulizer coupling for such a device in accordance with the present invention will now be described, by way of example, with reference to the accompanying drawings, in which:

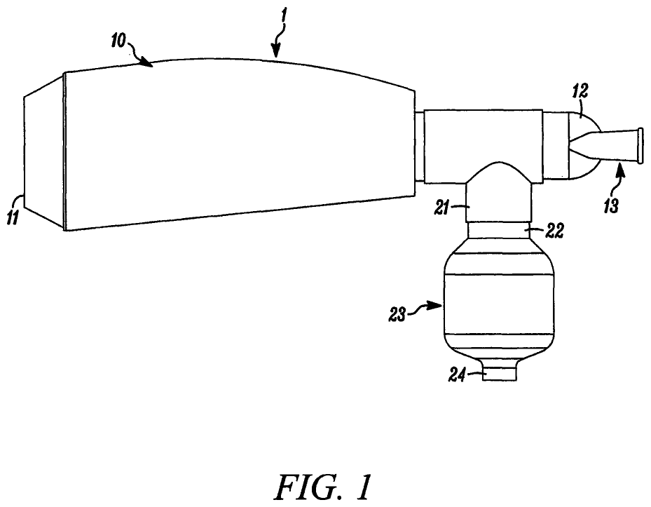

FIG. 1 is a side elevation view of an assembly of the device and a nebulizer;

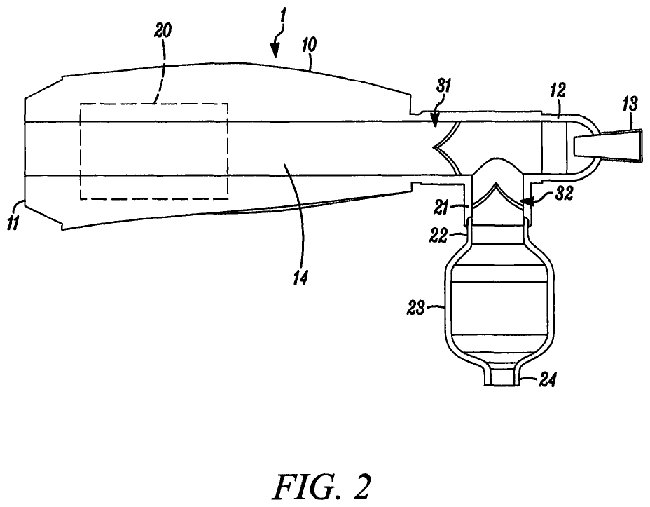

FIG. 2 is a cross-sectional side elevation view of the assembly;

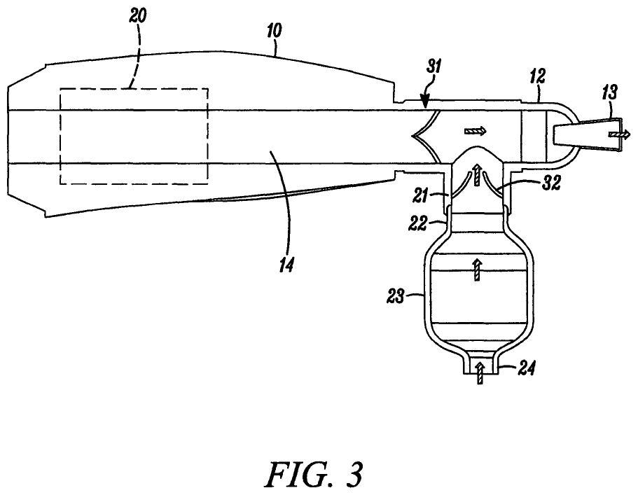

FIG. 3 is a cross-sectional side elevation view of the assembly showing an inspiration phase;

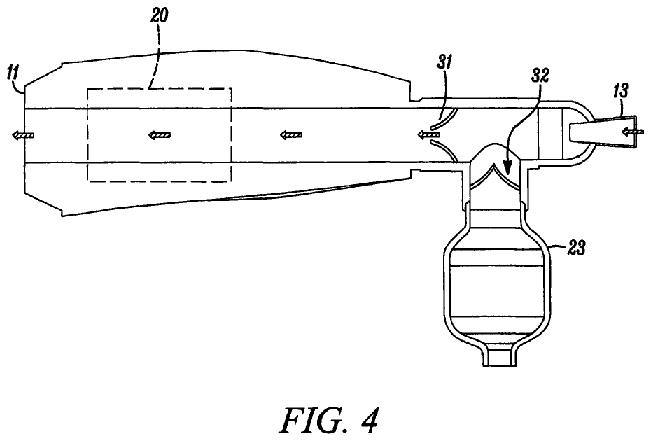

FIG. 4 is a cross-sectional side elevation view of the assembly showing an expiratory phase;

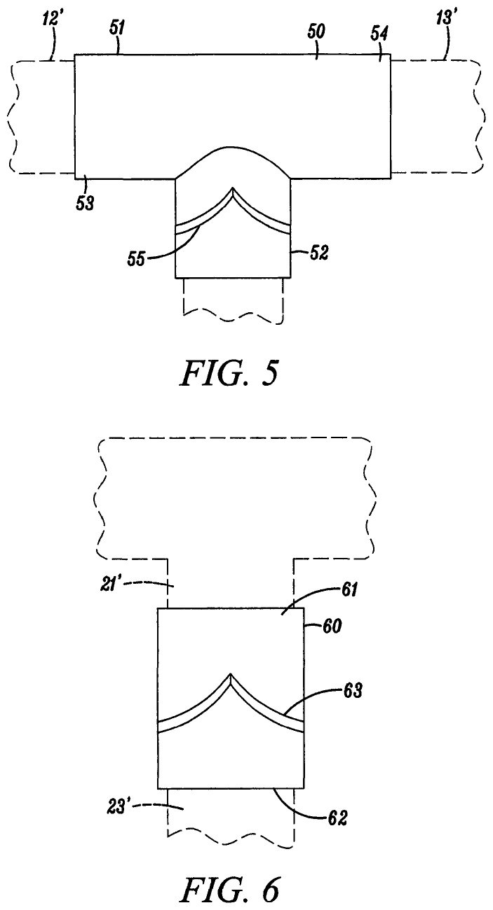

FIG. 5 is a cross-sectional side elevation view of a first form of coupling for fitting with a therapy device; and

FIG. 6 is a cross-sectional side elevation view of a second form of coupling for fitting with a therapy device.

With reference first to FIGS. 1 and 2, the device 1 has an outer transparent housing 10 with an expiratory opening 11 at its left-hand end and a patient inlet 12 and a mouthpiece 13 (or other patient interface) at its opposite, right-hand end. An air-flow path 14 extends between the expiratory opening 11 and the patient inlet 12. The device also includes a conventional mechanism indicated generally by the numeral 20 located along the air flow path that provides an alternating, oscillating or vibrating resistance to expiratory flow. Details of the operation of the mechanism 20 are not needed for an understanding of the present invention but, in brief, the mechanism includes a pivoted rocker arm supporting a conical valve member at one end that closes a trumpet-shape opening through Which expiratory air flows. The arm is lifted by air flow and then drops down to close the opening, this being repeated continuously during expiration. Further details of the mechanism can be seen in U.S. Pat. No. 6,581,598. Prior devices also include an air inlet path so that the patient can inhale without having to remove his mouth from the device. In some devices this airflow path opens directly to atmosphere via a one-way valve that enables inspiration but prevents expiration, so that all expiratory flow is through the oscillatory mechanism. Devices provided with a nebulizer coupling have an inhalation path that is uninterrupted to the nebulizer outlet. In the device shown in FIGS. 1 and 2 the housing 10 has a nebulizer port 21 projecting outwardly of the housing at right angles to the axis of the device and opening into the air flow path 14 close to the patient inlet 12. The nebulizer port 21 receives the outlet 22 of a conventional nebulizer 23 (shown only schematically in the drawings). The therapy device 1 and the nebulizer 23 together provide the respiratory therapy assembly. The nebulizer 23 contains a fluid, typically with a medicine, and receives a supply of pressurized gas at its inlet 24 to create a mist of vapour that is available at its outlet. The nebulizer may also be heated by power from an external power supply (not shown).

The invention could be used with other oscillatory respiratory therapy devices having a different form of mechanism for producing an oscillating resistance to expiration.

The therapy device 1 also includes two one-way valves 31 and 32. The first valve 31 is located in the air flow path 14 between the oscillatory mechanism 20 and the nebulizer port 21. The valve 31 may be a duck-bill, flap or similar valve and is arranged to allow expiratory flow (from right to left in the drawings) along the flow path 14 but to prevent or substantially restrict flow in the opposite direction. In this way, the user can exhale along the air flow path 14 and open the valve 31 but when he attempts to inhale the valve closes and prevents any airflow (or a major part of it) from that part of the air flow path on the side of the valve away from the patient.

The second valve 32 may be of the same type as the first valve 31, or could be a different form of valve, but is located in the nebulizer port 21 and is oriented such that air and nebulizer vapour produced by the nebulizer 23 can flow from the port into the air flow path 14 between the patient inlet 12 and the first valve 31. The second valve 32 is continuously exposed to positive pressure on its lower, nebulizer side from gas supplied to the nebulizer inlet 24. The second valve 32 is, therefore, arranged such that it is not opened by this positive pressure from the nebulizer alone but is only opened when the differential pressure across the valve rises above this as a result of negative pressure on its upper side caused during inhalation. The second valve 32 is, therefore, arranged so that it is only opened during an inhalation phase.

FIG. 3 shows path of gas flow during an inhalation phase where it can be seen that the first valve 31 is closed by the reduced pressure created at the patient inlet 12. The second valve 32 in the nebulizer port 21 is opened allowing air and nebulizer vapour to flow from the nebulizer 23 into the air flow path 14 on the patient side of the first valve 31 and from there can be inhaled by the patient so that the vapour passes into his respiratory passages.

FIG. 4 shows what happened when the patient exhales. This creates an increased pressure at the patient inlet 12 and hence in the right-hand, patient end part of the air flow path 14. This increased pressure closes the second valve 32 in the nebulizer port 21 and opens the first valve 31, thereby allowing air to flow along the air flow path 14 to the oscillatory mechanism 20. During this expiratory phase the second valve 32 in the nebulizer port 21 closes so that vapour produced in the nebulizer 23 is trapped in the nebulizer and is not wasted by being exhausted to atmosphere during exhalation.

The invention could also be used in a therapy device that does not have any built-in nebulizer coupling by using a separate coupling 50 of the kind shown in FIG. 5. This coupling 50 has a T-shape comprising a straight tubular main arm component 51 with a side arm 52 projecting outwardly from the main arm midway along its length. One end 53 of the main arm 51 is shaped to be fitted onto the patient inlet 12′ of the respiratory device and its opposite end 54 is shaped to receive a mouthpiece 13′ or other patient interface. The side arm 52 contains a one-way valve 55 arranged in the same manner as the valve 32 of the device described with reference to FIGS. 1 to 4, to allow nebulizer vapour to flow into the therapy device during an inhalation phase and to close during an expiration phase.

The invention could, furthermore, be used with therapy devices that have a conventional (unvalved) nebulizer port either by incorporating the nebulizer outlet one-way valve in a part of the nebulizer itself or by means of a separate coupling 60 of the kind shown in FIG. 6. This coupling 60 is a simple tubular fitting where one end 61 is adapted to fit onto the end of the conventional nebulizer port 21′ and its opposite end 62 is adapted to receive coupled to it the outlet of a nebulizer 23′ so that the coupling is positioned between the nebulizer and the therapy device. The coupling 60 contains a one-way valve 63 arranged in the same manner as the valve 32 of the device described with reference to FIGS. 1 to 4, to allow nebulizer vapour to flow into the therapy device during an inhalation phase and to close during an expiration phase.

The arrangement of the present invention reduces waste of nebulizer fluid and medication and enables a given volume of medication to be administered over a longer period. By reducing waste the clinician has a better knowledge of the actual amount of nebulizer medication utilized by the patient.

1-10. (canceled)

11. An oscillatory respiratory therapy device with a patient inlet and a mechanism arranged to produce an oscillating resistance to breathing through the inlet, characterised in that the device includes a first one-way valve located between the patient inlet and the mechanism and arranged to allow expiratory flow from the patient inlet to the mechanism but to restrict flow through the valve in the opposite direction, that the device also includes a nebulizer port opening into the device between the patent inlet and the first valve, that the nebulizer port is adapted for connection with the outlet of a nebulizer, and that the device includes a second one-way valve arranged to allow flow from the port to the patient inlet during inhalation but to restrict flow from the port during exhalation.

12. A therapy device according to claim 11, characterised in that the mechanism includes a displaceable member in the form of a pivoted rocker arm supporting a valve member that closes and opens an opening through which expiratory air flows.

13. A therapy device according to claim 11, characterised in that first valve is a duck-bill valve.

14. A therapy device according to claim 11, characterised in that the second valve is a duck-bill valve.

15. A therapy device according to claim 11, characterised in that the second valve is provided on a coupling separate from and removably connected to the therapy device, and that the coupling provides a connection for the outlet of a nebulizer.

16. A therapy device according to claim 15, characterised in that the coupling also provides a connection for a patient interface.

17. An assembly of a respiratory therapy device according claim 11, and a nebulizer connected with the nebulizer port.

18. An assembly of a nebulizer and an oscillatory respiratory therapy device, the therapy device having a patient inlet and a mechanism arranged to produce an oscillating resistance to breathing through the inlet, characterised in that the assembly includes a first one-way valve located between the patient inlet and the mechanism and arranged to allow expiratory flow from the patient inlet to the mechanism but to restrict flow through the valve in the opposite direction, that the assembly also includes a nebulizer port opening into the device between the patent inlet and the first valve, that the nebulizer port is connected with the outlet of a nebulizer, and that the assembly includes a second one-way valve arranged to allow flow from the nebulizer to the patient inlet during inhalation but to restrict flow from the nebulizer during exhalation.

19. A nebulizer coupling for fitting to the patient inlet of an oscillatory respiratory therapy device, characterised in that the coupling includes a one-way valve arranged to allow flow from the coupling to the patient inlet during inhalation but to restrict flow from the coupling during exhalation.

20. A nebulizer coupling according to claim 19, characterised in that the coupling also provides a connection for a patient interface.
